(19) United States
(12) Patent Application Publication
(10) Pub. No.: US 2026/0215688 A1
(43) Pub. Date: Jul. 30, 2026

(54) SYSTEMS, DEVICES AND METHODS FOR DYNAMIC POLARIZED LIGHT IMAGING

(71) Applicant: NerView Surgical Inc., Hamilton (CA)

(72) Inventors: Anish S. Naidu, Hamilton (CA); Hilary Luo, Hamilton (CA)

(21) Appl. No.: 19/462,656

(22) Filed: Jan. 28, 2026

Related U.S. Application Data
(60) Provisional application No. 63/750,470, filed on Jan. 28, 2025.

(51) Int. Cl. A61B 5/00; G06T 7/00

(52) U.S. Cl. CPC A61B 5/0077; A61B 5/0082; A61B 5/4029; A61B 5/743; G06T 7/0012

(57) ABSTRACT

Provided is a system, method and device for dynamically changing polarized light imaging to identify optically anisotropic structures in the field of view, such as nerves, in real-time. A specific application of this system is for use during surgery in order to distinguish nerves from other tissues. The system includes a light output system including one or more light sources that dynamically produces polarized light; and an imaging detection system including one or more image detectors that captures images of a target; and a capture synchronization and control system that manages electrical and electromechanical adjustments of the light output system and the imaging detection system, to dynamically adjust the light sources and image detectors to synchronize with each other.

### TECHNICAL FIELD

The embodiments disclosed herein relate to imaging in the medical field, in particular to the real-time imaging of organic tissues.

### INTRODUCTION

Identification of nerves in the surgical field may assist surgeons in recognizing and protecting nerves from unintentional damage during surgery. The application may be adapted for use in open surgery, minimally-invasive surgery, robot-assisted surgery, microscope-assisted surgery, and endoscopic procedures. The technique may also be adapted for use with other tissues or inorganic objects, so long as they have an optical response that varies depending on the polarization angle of the incident light, and hence the dynamically-changing polarized light imaging technique can produce useful data beyond imaging with unpolarized light or fixed polarized light.

One major application of the technology described herein is to assist surgeons in the real time identification of nerves in the surgical field. Nerves are very important structures in the body, and are responsible for the communication of signals between the central nervous system (CNS) and the rest of the body. The CNS includes the brain and the spinal cord. Nerves may either be of motor type, which carry signals from the CNS to structures like muscles to make them move, or of sensory type, which carry signals from receptors like skin touch, pain, temperature and joint position to the CNS for feedback.

Damage to the nerves can have minor to devastating consequences depending on the specific nerve and the severity of damage. For example, damage to a nerve supplying a muscle effectively results in the paralysis of that muscle as the brain can no longer control it, or damage to a nerve from a patch a skin makes that area feel numb. Some nerves are more critical, such as those around the throat, as damage to them can result in loss of speech and the ability to swallow.

The extent of damage to the nerve may be variable depending on the cause. Broadly, mechanical damage may be either those where the nerves are cut, or those where the nerves are crushed of stretched. If the damage is minor, it is possible that the nerves can heal and the connections can regrow over days to weeks, and the functionality may be restored. If the damage is major, the functionality may be lost forever.

There are surgical options for the treatment of nerve damage that is not expected to heal on their own, however the success rate is variable. The options include trying to rejoin a cut nerve, using a graft to replace damaged nerve, or transferring a nerve from one location to another. These procedures are challenging and specialized, making them expensive. The ideal option is to avoid nerve damage in the first place.

There are various causes of damage to nerves, with common ones being accidental trauma, medical illnesses, and accidental damage during surgeries. Of these, an accidental damage during a surgery is the most preventable. Data shows that 10-20% of all nerve injuries are caused accidentally during surgeries. Such injuries can have a significant negative impact on a patient's recovery and quality of life.

Currently, there are limited options available to surgeons to assist them in identifying nerves in real time during surgery, so that they may protect them from accidental damage. In a surgical field, the nerves, especially the smaller ones (<2 mm), may be quite hard to find and distinguish from the other tissues around them, such as blood vessels, fat and connective tissue. The nerves just look like white strands to the untrained eye. Surgeons mainly depend on their knowledge of anatomy and experience to identify the nerves with their naked eye.

For certain procedures with critical nerves, the current standard of care is using electrical stimulation and detection. This approach has limitations such as only specific nerves may be monitored, requires the insertion or application of electrodes before or during surgery, and does not actually display the nerve but rather signals if the surgeon comes close to the nerve or moves the nerve. There are upcoming technologies that involve the use of a fluorescent dye that is administered to the patient intravenously prior to the surgery, and the dye molecules attach to the nerves and make the nerves glow when illuminated under specific wavelength of light during the surgery. This approach also has limitations such as requiring patient preparation before the surgery, and the administration of a chemical to the patient which can have adverse effects.

The benefits of the technology described herein are that it may be non-invasive to the patient and may not need any specific patient preparation prior to the surgery. It does not need administering any drugs to the patient, or any component of the system to contact the patient. The application works optically from a distance, and offers the flexibility to the surgeons to use it as and when needed.

Accordingly, there is a need for dynamically changing polarized light imaging methods, devices and systems described herein.

### SUMMARY

Provided herein is a system for dynamic polarized light imaging, the system comprising: a light output system including one or more light sources that dynamically produces polarized light with varying polarization angles, wavelengths and/or focus; an imaging detection system including one or more image detectors that captures images of a target under varying polarization angles, wavelengths and/or focus; a capture synchronization and control system that manages electrical and electromechanical adjustments of the light output system and the imaging detection system, to dynamically adjust the light sources and image detectors to synchronize with each other; and an image processing system that processes a plurality of images captured to compute and produce a final output.

In an embodiment, the light output system includes a polarization system to vary the polarization angle of the light produced, using a discrete polarization angle approach or a continuous variable polarization angle approach.

The light output system may include a plurality of reflectors to efficiently reflect the polarized light towards the target in a focused manner.

The light output system may include a plurality of lenses to adjust angles of spread and/or focus of the polarized light match the field of view and/or distance to the target.

The light output system may include a plurality of optical filters to restrict the polarized light to specific wavelength spectrum.

The light output system may include a beam-joining system for emitting the polarized light from a common output.

The image detection system includes a polarization system to vary the polarization angle of the light captured, using a discrete polarization angle approach or a continuous variable polarization angle approach.

The image detection system may include a polarization system that differs from the polarization system in the light output system.

The image detection system may record reflected light with a distribution of polarization angles from the target being imaged in a field of view.

The imaging detection system may include a plurality of optical filters for tuning to different light wavelengths.

The imaging detection system may include a plurality of lenses that adjustably focus the reflected polarized light onto the plurality of imaging sensors to view the target at different fields of view and/or distances.

The imaging detection system may include a plurality of imaging sensors for capturing multiple images that are fused and processed for detecting optically anisotropic structures of interest, such as nerves, in the field of view.

The plurality of imaging sensors may be configured to independently capture the reflected polarized lights from the field of view with their own lenses and optical filters.

The imaging detection system further may include a beam-splitting system for directing light from a single point-of-view to the plurality of imaging sensors.

The polarization system included in the imaging detection system may capture a plurality of images of the target at different polarization angles.

The capture synchronization and control system may maintain the relationship between the polarization angles and other parameters of the light output system and the image detection system, using an approach related to electrical linking, mechanical linking, or software control.

The system further may include an image processing system for processing a plurality of images of the target captured in the field of view under different polarization angles and different light wavelengths, such that structures that are optically anisotropic appear with different brightness in the plurality of images.

The image processing system may spatially synchronize the plurality of images for making determinations about the structure at a certain pixel from data captured, such that each corresponding pixel in the images refer to the same point in the field of view.

The image processing system may segment and identify structures of interest in the field of view.

The system may further include or connect to an output device for highlighting and displaying the structures of interest in the field of view.

The final output that is displayed may identify the structures of interest in the field of view using a color overlay or annotation, and may further specify a confidence level to indicate the certainty that a given labelling is accurate.

The light output system, the imaging detection system and the capture synchronization and control system may be integrated into an imaging device.

The imaging device may be a handheld device including an ability to optionally attach the imaging device to a hands-free mounting system.

The handheld imaging device may include a handle that may form an acute (<90 degrees) angle with respect to the head, which may allow for the handheld device to be more comfortably pointed down towards a surgical field.

The imaging device may be attached to or integrated into a surgical microscope.

The imaging device may be attached to or integrated into an endoscope.

The system may include a motion tracking system for tracking translational and rotational motion as well as a distance of the imaging device from the target being imaged.

The capture synchronization and control system may execute a feedback loop with independent monitoring sensors to change the polarization angle, focus, or other imaging parameters in a precise and coordinated manner.

The system may perform ambient light compensation by estimating an ambient light intensity to adjust for dynamic variations of ambient lighting conditions.

The system may perform distance compensation by estimating the distance to a target when the distance of the light output system or the imaging detection system from the target is varied during use.

The system may perform motion compensation by estimating the motion of the imaging device for spatially aligning the plurality of images when the imaging device is moved during use.

The system may perform target surface orientation compensation using a plurality of light outputs and/or imaging detectors to capture images under different lighting perspectives.

The system may integrate image data acquired by at least one manner of regular visible light color imaging, laser speckle contrast imaging (LSCI), near-infrared (NIR) imaging, hyperspectral imaging, fluorescent dye imaging, autofluorescence imaging, and Raman scattering imaging into the dynamic polarized light imaging, to identify and distinguish various structures.

Provided herein is a method for image data processing, the method comprising: pre-processing a plurality of image data of a target captured in a field of view for image correction and enhancement by algorithms; spatially aligning the plurality of image data for making determinations about structures of interest at a certain pixel from the data captured; fusing the plurality of image data to detect varying brightness of pixels and highlight structures of interest at different angles in the field of view; detecting and segmenting the fused image data and pre-fusion image data to label features of interest in the field of view; tracking data related to translational and rotational motion, and distance of an imaging device from the target being imaged to support the alignment among captured images; post-processing the segmented and labelled images for improving quality; and displaying a visually-appealing final output with the structures of interest highlighted and labelled.

The image data may include data related to ambient light compensation, target distance compensation, and target surface orientation compensation to improve output accuracy.

In one embodiment, the method may further include providing additional data to improve output performance by integrating supplemental imaging modalities.

Provided herein is an imaging apparatus for dynamically changing polarized light imaging, the imaging apparatus comprising: a handle for providing a grip to facilitate a user in directing the imaging apparatus downward towards a field during a surgery; a head for employing a plurality of components, wherein the plurality of components further includes: a light output system including a plurality of infrared (IR) light sources with dynamically changing polarization located at up to four corners of the front view of the head, using mechanical rotation of a polarizing filter approach, and a white light source in the middle at the top or bottom of the front view of the head; an imaging detection system including an IR camera located at the center of the front view of the head, also with dynamically changing polarization, and mechanically synchronized to the light sources to maintain a 90° offset, and a visible spectrum RGB camera in the middle at the top or bottom of the front view of the head; and a capture synchronization and control system configured to manage electrical and electromechanical adjustments of the light output system and the imaging detection system, to dynamically adjust the light sources and image detectors to synchronize with each other; a plurality of buttons for supporting the user to control the imaging device, and a plurality of indicator lights for presenting information to the user.

Provided herein is a system for real-time image processing, the system comprising: a base station configured to provide a plurality of functions, including image data processing, power source, charging station, light source, data output, and data recording, wherein the image data processing at least includes fusing images from the varying polarization angles, wavelengths and/or focus; an imaging device configured to capture a plurality of images of a target in a field of view under the varying polarization angles and varying light wavelengths, such that structures that are optically anisotropic appear with varying brightness in the plurality of images and structures of interest are highlighted; and wherein the base station is communicatively connected to the imaging device, and a plurality of output hardware.

Provided herein is a method of dynamic polarized light imaging, the method comprising: producing dynamically changing polarized lights from at least one or more light sources; capturing, by at least one or more image detectors, a plurality of images of a target and recording reflected light from the target being imaged in a field of view, wherein the reflected light includes a distribution of polarization angles; managing, by a capture synchronization and control system, electrical and electromechanical adjustments available on both the light output system and the imaging detection system, in order to dynamically adjust the image detectors to synchronize with the light outputs; and processing, by an image processing system, a plurality of images of the target under different polarization angles and different light wavelengths, such that structures that are optically anisotropic appear with varying brightness in the plurality of images and the structures of interest are highlighted.

Provided herein is a method of image processing of dynamic polarized light imaging, the method comprising: spatially aligning a plurality of images; executing a fusion of image data to detect varying brightness and highlight structures of the target at different angles in the field of view; detecting and segmenting the fused and pre-fusion image data to label features of interest in the field of view; post-processing the segmented and labelled images for improving quality; and generating a visual output to display and annotate the structures of the interest in the field of view.

Other aspects and features may become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a block diagram of a system for dynamic polarized light imaging, according to an embodiment;

FIG. 2 is a block diagram of a light output system for dynamic polarized light imaging, according to an embodiment;

FIG. 3 is a block diagram of an imaging detection system for dynamic polarized light imaging, according to an embodiment;

FIGS. 4A to 4D are example methods for dynamically changing polarization for the light output system and the imaging detection system, according to an embodiment;

FIGS. 5A to 5C are example methods for capture synchronization between the light output system and the imaging detection system, according to an embodiment;

FIG. 6 is an example method for imaging processing, applied in one or more image detectors, according to an embodiment;

FIG. 7 is an example method for imaging processing system, according to an embodiment;

FIGS. 8A and 8B are example functional components of the system, according to an embodiment;

FIGS. 9A to 9C are example devices employing the light output system, the imaging detection system and the capture synchronization and control system for dynamically changing polarized light imaging, according to an embodiment;

FIGS. 10A to 10D are photographs of an example device employing the light output system, the imaging detection system and the capture synchronization and control system for dynamically changing polarized light imaging, according to an embodiment;

FIGS. 11A to 11D are example raw captured images and final outputs highlighting the detected nerves, according to an embodiment; and

FIG. 12 is a block diagram illustrating a computing device used in the system for dynamic polarized light imaging.

### DETAILED DESCRIPTION

Various apparatuses or processes may be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

One or more systems described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, an embedded microcontroller, a single-board computer, a mainframe computer, server, personal computer, cloud-based program or system, laptop, personal data assistant, cellular telephone, smartphone, or tablet device.

Each program is preferably implemented in a high-level procedural or object-oriented programming and/or scripting language to communicate with a computer system. However, the programs may be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein.

A description of an embodiment with several components in communication with each other does not imply that all such components are required. On the contrary, a variety of optional components are described to illustrate the wide variety of possible embodiments of the present invention.

Further, although process steps, method steps, algorithms or the like may be described (in the disclosure and/or in the claims) in a sequential order, such processes, methods and algorithms may be configured to work in alternate orders. In other words, any sequence or order of steps that may be described does not necessarily indicate a requirement that the steps be performed in that order. The steps of processes described herein may be performed in any order that is practical. Further, some steps may be performed simultaneously.

When a single device or article is described herein, it may be readily apparent that more than one device/article (whether or not they cooperate) may be used in place of a single device/article. Similarly, where more than one device or article is described herein (whether or not they cooperate), it may be readily apparent that a single device/article may be used in place of more than one device or article.

Nerve cells (neurons) may exhibit anisotropic optical properties, owing to the microtubules as well as the myelin sheath oriented axially along the axons of the neurons. The nerve bundles are composed of axons along with myelin sheaths and connective tissue, making the nerves also exhibit birefringence properties. This means that the nerves interact with linearly polarized light differently than other tissues depending on the angle of polarization of the incident light relative to the axis of the nerve.

Specifically, polarization of the light that reflects off of the nerves is rotated based incident angle of polarization. Not all of the reflected light is rotated precisely as there are other connective tissues around the nerves that diffuse the polarization. Therefore, there is a distribution of polarization angles in the reflected light. This reflected light may be filtered and analyzed to distinguish nerves from other tissues.

An underlying challenge of this imaging principle, is that the polarization of the reflected light is directly dependent on the relative angle between the nerve axis and the polarization of the incident light. This means that in order to detect nerves at any angle in the field of view, the target area in the field of view is imaged using varying polarization angles.

To accomplish this, the light source in the imaging device may be able to produce light at adjustable polarization angles, and the image detector also may be able to filter the reflected light at adjustable polarization angles.

Best results may be obtained when the polarization angles of the light source and the detector are orthogonal (at 90°) relative to each other. This may minimize glare from objects in the field of view. This may make the nerves stand out more compared to other tissues.

The intensity of the reflected light measured by the detector is the highest when the nerve axis is at 45° to both the source and detector polarization angle, and the brightness decreases if the nerve axis moves away from 45 degrees. This specific property is unique to nerves and a few other tissues (such as muscle fibers and tendons), and most other tissues do not behave in this exact manner.

By imaging the same area under different polarization angles, the change in relative intensity of the detected light may be correlated with the angle of the structures in the field of view to distinguish nerves from most other tissues. In order to distinguish nerves for other structures that also interact with polarized light, further image processing may be applied. These techniques can use the size, shape, and orientation of the structures to isolate nerves. For example, nerves are thread-like structures, whereas muscles are band-like and tendons are rope-like.

Additionally, nerves and other tissues also behave differently when illuminated under different light wavelengths. Different tissues absorb and reflect a different portion of the light spectrum. For example, nerves tend to reflect more blue light than muscles and blood. The wavelength of the light also dictates the penetration depth. Blue light penetrates tissues to only about 2 mm whereas infrared light can penetrate to about 5 mm, for example. By analyzing the wavelength spectrum of the reflected light for different spectrums of incident light, additional useful information about tissue type and depth may be extracted.

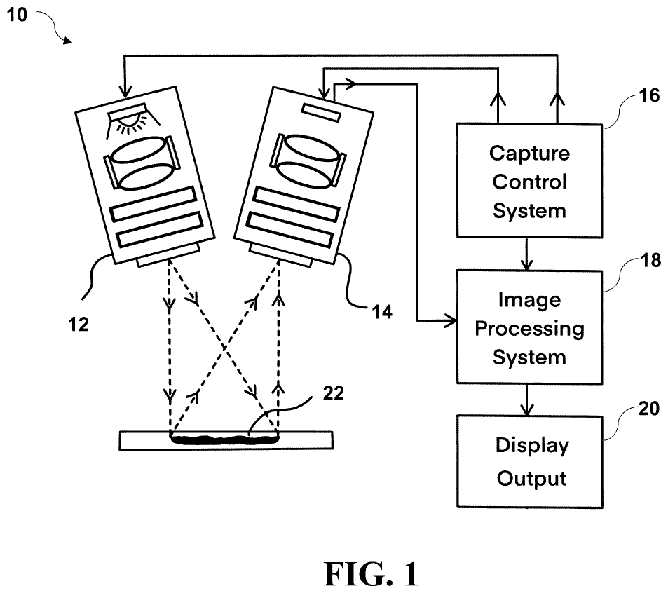

Referring now to FIG. 1, shown therein is a block diagram of a system 10 for dynamic polarized light imaging, according to an embodiment.

The system 10 includes a light output system 12 for providing illumination. The system 10 includes an imaging detection system 14 for capturing images. The system 10 includes a capture synchronization and control system 16. The system 10 includes an image processing system 18. The system 10 includes a display output 20. The system 10 is intended to image a target structure 22.

System 10 may include one or more light output systems 12 and one or more imaging detection systems 18. The additional components of the system 12 may be subject to variation depending on the application.

The light output system 12 produces light with controlled wavelength and polarization angles. The light output system 12 creates a light output with adjustable angles of polarization depending on the form factor and exacts use case in which this technology is being applied. The light output system 12 may restrict the lights to specific wavelength spectrum. This is matched with the wavelength spectrum that the imaging detection system 14 is tuned to. Furthermore, this enables imaging the target 22 illuminated under specific wavelengths of light and using this to identify the structures.

The light output system 12 focuses or collimates the light to direct the majority of the light towards the intended target 22. This enables more energy to reflect off of the target tissue and reach the imaging detector in a focused manner, enhancing the quality of data. The light output system 12 may have an adjustable focusing system. This may also enable the system 10 to be robust against other light sources around where the device is being used, such as operating room lights and endoscope lights.

The imaging detection system 14 includes one or more image sensors 302. The one or more image sensors 302 record a reflected light with a distribution of polarization angles from a target 22 being imaged in a field of view. The light reflected from the tissues being imaged is recorded for further image processing.

The imaging detection system 14 captures a plurality of images of the target 22 in the field of view. The data captured from the imaging detection system 14 may be precisely synchronized with the parameters of the light output system 12 at the moment when the data is captured.

The imaging detection system 14 includes a plurality of imaging sensors 302 for capturing images for detecting nerves and other structures of interest in the field of view. The imaging detection system 14 adjustably focuses the polarized lights onto the plurality of imaging sensors 302 to view the target at different distances. The imaging detection system 14 may tune to different light wavelengths.

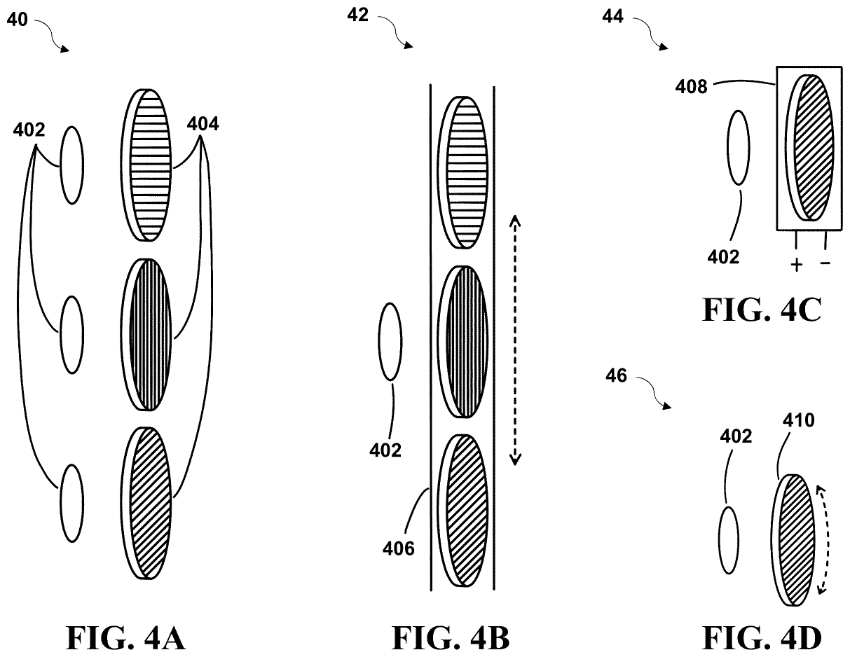

The system 10 may have a plurality of light outputs 12 and image detectors 14, tuned to different parameters. The light output system 12 and imaging detection system 14 may use different polarization system approaches in the same device, as shown in FIGS. 4A-4D.

The light output system 12 and the imaging detection system 14 are synchronized. This means that as the light output system 12 is being dynamically adjusted, the image detection system 14 may also need to be adjusted (depending on the specific choice of detector system) to match the light output. The synchronization involves matching of the light polarization angle and possibly the field of view, focal length, or light wavelengths.

The capture synchronization and control system 16 manages the electrical and electromechanical adjustments available on the light output system 12 and the image detection system 14, as well as triggers data capture from the imaging sensors at the appropriate time, to dynamically maintain the relationship between the polarization angle of the light output 12 and the polarization angle of the image detector 14. The capture synchronization and control system 16 may be running on an embedded microprocessor on an imaging device.

Further, the capture synchronization and control system 16 correlates the data captured with the settings on the light output system 12 and the image detection system 14, and forwards the data sets to the image processing system 18.

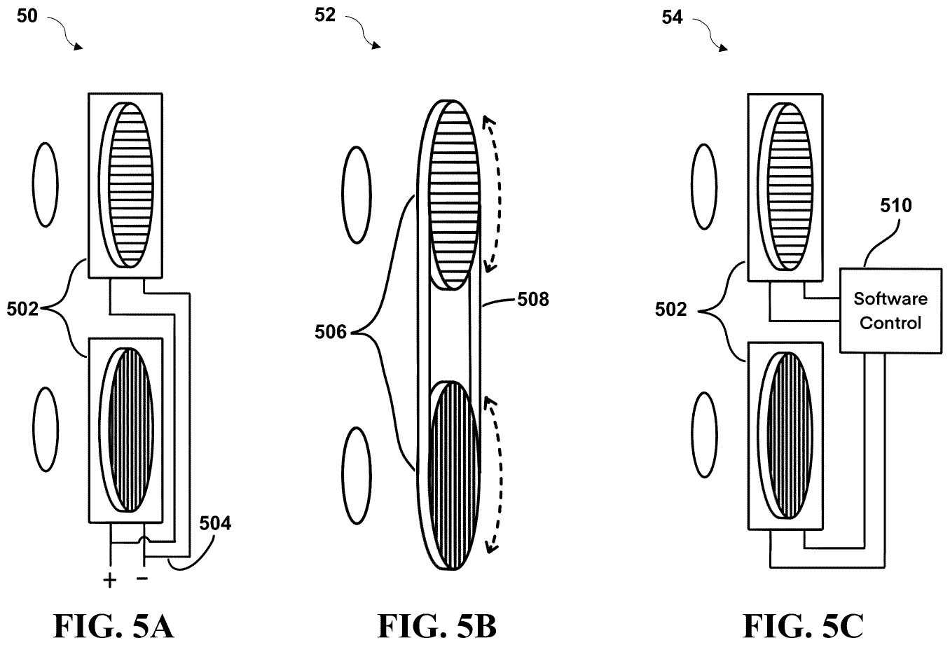

The synchronization of the light output system 12 and the image detection system 14 may be accomplished by the capture synchronization and control system 16 using one or a combination of the manners, such as, electrically linked, mechanically linked, and software controlled, as shown in FIGS. 5A-5C.

Additionally, the data captured from the image detection system 14 is precisely synchronized with the settings of the light output system 12 and the image detection system 14 at the moment when the data is captured. The polarization angle of the light output system 12 is known when a given image sample is captured. This information is used for the image processing system 18 and correlates the intensity of the light detected from objects in the field of view to the angle of polarization of the light output system 12 and the image detection system 14.

In order to be aware of the polarization angle, the capture synchronization and control system 16 may have to use a feedback loop with independent monitoring sensors depending on the approach used to change the polarization angle. In case of discrete polarization angle approaches, monitoring sensors are not typically used since the control system may specifically direct the transition to each discrete polarization angle. Hence the control system may inherently know the active polarization angle. This is the role of the capture synchronization and control system 16, and enables the image data processing system to identify nerves vs other tissues.

The image processing system 18 pre-processes a plurality of images of the target 22 captured in the field of view for image correction and enhancement by algorithms.

The image processing system 18 spatially synchronizes the plurality of images for making determinations about the structure at a certain pixel from data captured, such that each corresponding pixel in the images refer to the same structure in the field of view, such as behaviors at different polarization angles or wavelengths of light output.

The image processing system 18 may also use the data from the capture synchronization and control system 16 to increase the accuracy and speed up the process of spatial synchronization.

The image processing system 18 fuses the plurality of images to highlight structures at different angles in the field of view. Different algorithms may be employed to fuse the images based on the data from the capture synchronization and control system 16. The angle of polarization at which the images are captured is taken into consideration when fusing the data in order to highlight structures at different angles in the field of view. Machine learning techniques may also be employed to fuse the data and extract features of interest.

The image processing system 18 further segments and identifies structures of interest in the field of view. One or more types of structures in the field of view may be labelled depending on the application. The labeling may also have a confidence level that may be communicated to the user (graphically or textually) to indicate the certainty that a given labelling is accurate.

The image processing system 18 checks the segmented and labelled images to improve accuracy of labelling. A plurality of functions, such as filtering and removal of incorrectly labelled areas (using properties like size, shape, etc.), averaging of multiple samples to improve confidence level, smoothening of labelled regions may be performed by multiple algorithms, depending on the application.

The system 10 may further include a motion tracking system 732 for tracking transactional and rotational motion as well as a distance of the imaging device from the target 22 being imaged. The image processing system 18 may also use the data from the motion tracking system to increase the accuracy and speed up the process of spatial synchronization.

While capturing a plurality of images of the desired target under different polarization angles and/or wavelengths, it is possible that the imaging device may move. This is more likely in applications where the device is handheld, or there is vibration in the mounting apparatus. For proper image processing, the multiple images must be spatially aligned, i.e. each corresponding pixel in the images should refer to the same structure in the field of view. This may be achieved in software image processing via image registration approaches, however, this may be sped up and made more accurate by monitoring the motion of the imaging device.

By tracking the motion of the imaging device, including translational and rotational motion, as well as the distance of the imaging device from the target being imaged, the captured images may be spatially aligned. The measured motion may be used to compute a transformation per captured image which, when applied on the image, may assist in aligning the captured image to a reference image (such as the first image captured in the sequence).

Due to inaccuracies in any approach that may be used for measuring the motion of the imaging device, the transformation is not to be perfect, however, it may bring the images quite close to perfect alignment. Software image registration may be used to perfect the alignment of the images. The benefit of this approach is that the software image registration can run faster, more efficiently, and more accurately since the images are already quite close to perfect alignment.

Approaches may be employed for motion tracking and compensation depending on the application, including inertial tracking, optical tracking and electromagnetic tracking, and robotic tracking.

Inertial tracking includes an inertial measurement unit (IMU), which is a combination of 3-axis accelerometers, gyroscopes and magnetometers, placed in the imaging device that may be used to estimate translational and rotational motion. This is a compact and self-contained solution. Data from the different sensors in the IMU are filtered and fused to improve overall accuracy.

Optical tracking includes optical markers that may be placed on the imaging device and/or the surrounding environment. Cameras within or external to the imaging device may be used to detect and track the motion of the markers to estimate the motion of the imaging device.

Electromagnetic tracking includes an electromagnetic sensor unit placed in the imaging device and there is a fixed external electromagnetic field generator in the surrounding. The field generator creates a specific pattern of magnetic field, and the sensor in the imaging device is able to localize itself in the environment by analyzing the magnetic field detected.

Robotic tracking includes, in applications where the imaging device is affixed to an active or passive robotic arm, the arm that includes sensors, such as joint position sensors, that may be used to precisely calculate the location of the imaging device in the environment, and track its motion.

The system 10 may further include an ambient lighting compensation system to ensure optimal performance regardless of dynamic variations in ambient lighting conditions, and without requiring significant modifications to the operating environment, such as turning off the room lights. Approaches may be employed for immunity to the ambient lighting in the environment, including using non-visible wavelengths, and active compensation.

Using non-visible wavelengths involves the imaging device utilizing wavelengths outside the visible spectrum, such as infrared and ultraviolet, in order to have immunity against the ambient lighting, since the ambient lighting in a room is in the visible spectrum in general. The light output system 12 may be set to emit only the desired non-visible wavelengths by using specific sources 202 or wavelength filters 208, and the imaging detection system 14 may be include wavelength filters 306 that block out the visible light and only respond to those specific non-visible wavelengths. An additional benefit of this approach is that the non-visible light output from the imaging device will not interfere with the vision of the users of the device, or the function of other nearby optical systems operating in the visible light spectrum. This is particularly important since dynamically changing polarization of visible light can cause a strobing effect in the visual field of the users which can be quite distracting depending on the intensity. Using wavelengths in the non-visible spectrum avoids this issue completely.

Active compensation involves the system 10 actively measuring the ambient lighting in the room and adjust imaging parameters, such as brightness, exposure, contrast and white balance, to compensate for the effects of the ambient lighting. The ambient lighting may be measured using the imaging sensors 302 themselves, or by using separate dedicated ambient light sensors.

The system 10 may further include a target distance compensation system. In some embodiments, the distance of the light output system 12 and the imaging detection system 14 from the target 22 being imaged may vary during use. For optimal performance, the compensation system may have to monitor this distance and make continuous adjustments to various imaging parameters and image processing steps. Approaches may be employed for measuring the distance, including autofocus techniques, and distance sensors.

Autofocus techniques involve the imaging detection system 14 employing techniques such as Contrast Detection Autofocus (CDAF) and Phase Detection Autofocus (PDAF) using the imaging sensor 302 and the adjustable focusing lenses 304. In these techniques, the focusing lenses are periodically gradually adjusted and the imaging sensor is used to monitor the image contrast and/or phase shift. When the contrast is at its peak and the phase shift is matched, the image is in focus. At this time, the position and focal length of the adjustable lenses can be used to calculate the distance to the target 22. The autofocus cycle can be set to automatically run if the imaging device is moved or if the captured images are detected to be out of focus.

Distance sensors involves the imaging device using one or more independent distance sensors, such as a laser range finder or an ultrasonic range finder, pointed towards the target 22 to determine the distance to the target. This approach generally results in a more accurate and faster distance measurement. Unlike autofocus based measurement, this technique allows the distance to be measured continuously independent of the imaging detector.

In addition, the distance measurement applies to several important uses, including but not limited to: light intensity adjustment, focus adjustment, exposure adjustment, parallax correction, motion compensation, and target size estimation.

The intensity of the light output system 12 may be adjusted based on distance to maintain consistent image illumination. If the light output has a divergent beam, the illumination per unit area on the target will decrease with increasing distance. This can be compensated by increasing the intensity of the light output for greater distance.

The focus of the light output system 12 and the imaging detection system 14 may be adjusted using the respective lenses 206 and 304 based on the distance to the target to ensure optimum image quality.

The exposure time of the imaging sensor 302 may be tuned based on the distance to the target to ensure optimal brightness and contrast of the images.

When a plurality of imaging detection systems 14 are being used to capture images of the target, the difference between the positions of the imaging detectors results in each detector capturing a slightly offset image of the target. This is a form of parallax error that has to be corrected to align the images from the different detectors. The offset amount between the images depends on the distance to the target. The offset is higher when the target is closer, and approaches zero as the target moves far away. Therefore, measuring the distance to the target enables more accurate parallax correction.

As described above, the multiple images captured by the imaging detection system 14 may have spatial shifts due to motion of the imaging device. The motion compensation system is used to detect this motion in order to compensate for the shift and spatially align the plurality of images. However, the same motion can cause different amounts of shift depending on the distance of the target, with a closer target resulting in greater shifts. Therefore, the distance to the target is an important variable required for the motion compensation system to function accurately.

The distance of the target from the imaging device can also be used by the image processing system to estimate the true size of the structures of interest in the images. The apparent size of structures in the images is larger when the target is closer. Information about true size can be used to gain additional insights about the structures in the field of view since different structures such as nerves, muscles, tendons and vessels have different sizes.

The system 10 may further perform target surface orientation compensation to improve the signal quality and obtain consistent results when imaging the target structure at different surface orientations.

Light has the property that when reflected off a surface, the reflected light is preferentially polarized in the direction parallel to the plane of the surface, and perpendicular to the plane of incidence and reflection. This effect is maximal at the Brewster's angle but is also present to a lesser extent at all angles. Conversely, this also means that the specular reflection or glare is variable depending on the polarization of the incident light. The reflection is greatest when the incident light is polarized perpendicular to the plane of incidence and reflection, and least when the incident light is polarized along the plane of incidence and reflection.

This dependence of the reflected intensity on the polarization of incident light due to the angle of the surface adds noise to the signal in the polarized light imaging technique. The polarization of the reflected light is desired to be dependent only on the structures in the target 22, not the orientation of the surface of the target.

In order to compensate for this source of noise and improve the signal quality, the images of the target 22 can be obtained from different orientations. This can be achieved using multiple light outputs 12, multiple imaging detectors 14, or both, where the plane of incidence and reflection of one set of light output and detector is different from the other. When using two sets of light outputs and detectors, the respective planes of incidence and reflection may be perpendicular for the best compensation. Using more sets of light outputs and detectors at equally spaced out planes of incidence and reflection may improve compensation.

When using multiple light outputs 12, different outputs can be turned on and off alternatingly to capture images from different lighting perspectives and then fused to cancel out the effect of the angle of surface of the target 22. As an added benefit, this technique of capturing and fusing images from different lighting perspectives may also help cancel out the glare from specular reflections.

The system 10 may integrate supplemental imaging modalities alongside the core dynamic polarized light imaging technology disclosed herein. These supplemental modalities may serve to provide additional data or independent additional visualization.

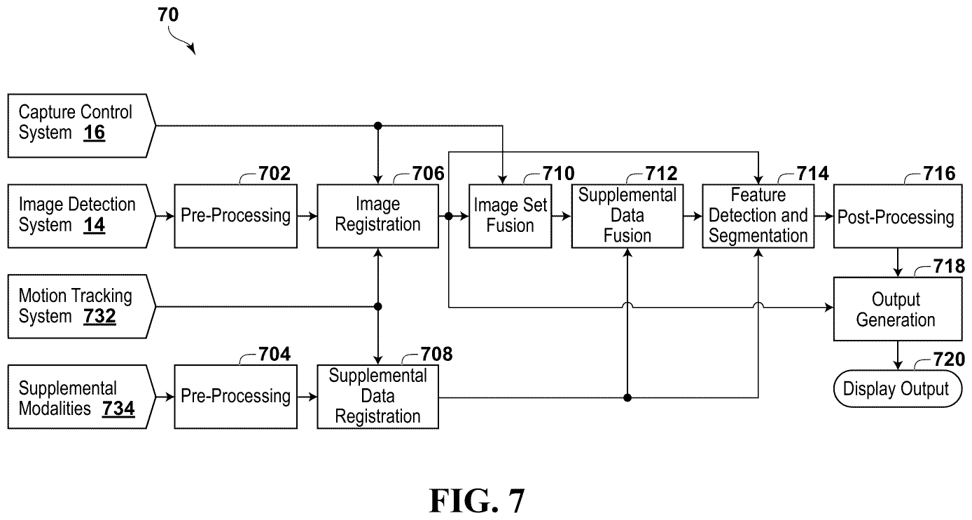

The supplemental modalities may provide additional data about the target being imaged such that this additional data may be integrated in the image processing (FIG. 7) to improve the accuracy with which the structures in the target are identified. In this case, the supplemental modalities may also need to be partially or wholly controlled by the capture synchronization and control system.

The supplemental modalities may provide independent additional visualization modes of the target, that provide the user with information about the target that is complementary to dynamic polarized light imaging. This may be primarily for the convenience of the user of this system, in that a single system may be capable of providing different imaging options to view the target depending on the application, as opposed to having to switch among multiple devices.

The supplemental imaging modalities may most likely also be optical imaging technologies, such that can also operate from a similar distance as dynamic polarized light imaging.

In some embodiments, technologies related to supplemental imaging modality include laser speckle contrast imaging (LSCI), near-infrared (NIR) imaging, hyperspectral imaging, fluorescent dye imaging, autofluorescence imaging, and Raman scattering imaging.

LSCI detects the variation in the interference pattern when the target is illuminated under a coherent light, typically to sense the motion of red blood cells representing blood flow. It can, therefore, provide a visualization of the perfusion of the target tissue being visualized. This can provide valuable information to integrate into dynamic polarized light imaging because different structures have different extent of perfusion. For example, vessels and muscles have significant perfusion, whereas tendons have minimal perfusion.

NIR imaging detects the presence of blood owing to the property that blood absorbs NIR light. When imaging a target illuminated under NIR light, structures like blood vessels that have significant concentration of blood, appear darker than the surrounding. This may be particularly helpful in distinguishing nerves from blood vessels.

Hyperspectral imaging employs simultaneous detection of a wide spectrum of light by breaking down the light striking each pixel into many different spectral bands instead of just red, green and blue as in typical color imaging. This provides more information on the exact spectrum of light reflected from the structures being imaged. When the spectrum of the incident light is known, the differences in the spectral pattern of the reflected light may be used to detect what structures are being imaged. This can provide valuable information to integrate into dynamic polarized light imaging to better identify and distinguish various structures like nerves, tendons, muscles and connective tissue.

Fluorescent dye imaging uses a fluorescent dye that is administered to the patient a certain time period before imaging. The dye is engineered to specifically bind to certain molecules in the tissues such that the dye specifically concentrates in an area of interest, such as in blood, bile, urine, nerve tissue, etc. When the target tissue is illuminated with a particular wavelength of light, the dye fluoresces, typically emitting light at a different wavelength. By imaging the target at that particular wavelength, structures that the dye was designed to bind to may be easily identified. This technique is currently widely used in surgery; however, it is not non-invasive as it has the injection of a dye. The primary reason for integrating this technology may be for the convenience of the user, as this single device can also provide the functionality.

Auto fluorescence imaging uses the property of certain biological tissues that they inherently fluoresce when illuminated by certain wavelengths of light, generally ultraviolet and blue-green light. The fluorescence response is at a higher wavelength then the wavelength used to illuminate the tissues. The exact wavelength and intensity of fluorescence varies for different tissue types, and this can be used to identify structures of interest. This can provide valuable information to integrate into dynamic polarized light imaging to better identify and distinguish various structures.

Raman scattering imaging uses the property of various chemical compounds that they absorb incident light at a particular wavelength and release low-intensity scattered light at a specific higher wavelength depending on the chemical bonds in the compound. For biological tissues, the imaging is generally performed using an incident beam of near infrared (NIR) light, and the scattered light is in the short-wave infrared (SWIR) region which requires special expensive cameras such as InGaAs sensors. Different tissues have different proportions of specific chemical bonds resulting in a characteristic wavelength distribution of scattered light that can be used to identify structures of interest. This can provide valuable information to integrate into dynamic polarized light imaging to better identify and distinguish various structures.

The system 10 further includes a display output 20 for highlighting and displaying the structures of interest in the field of view. A visually-appealing final output is generated to display the information desired by the user in an intuitive format. This may involve approaches such as taking the original image and using transparent color overlays to highlight specific structures of interest. Other graphical or textual annotations may also be added, such as specifying size measurements or confidence level. This final output may be ultimately what is displayed to the user.

The light output system 12 and the imaging detection system 14 may be integrated into an imaging device. The capture synchronization and control system 16 may be employed in an imaging device as well. The imaging processing system 18 and the display output 20 may be employed in a base station of an imaging device.

The aforementioned imaging technique may also be adapted for imaging tissues outside of a surgical setting or inorganic objects, as long as they have an anisotropic optical response that depends on the polarization angle of the incident light.

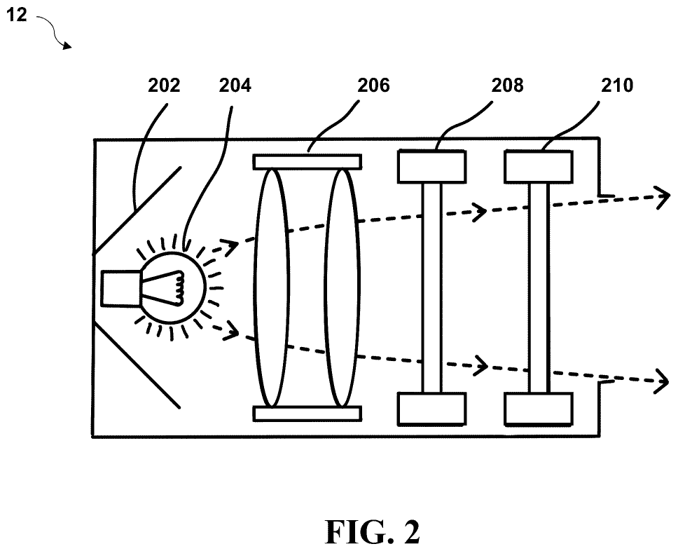

Referring now to FIG. 2, shown therein is a block diagram of a light output system 12 for dynamic polarized light imaging, according to an embodiment. The specific arrangement, configuration, form, dimensions (e.g. shape, size), layout of the components of the light output system 12 may be subject to variation depending on application. The light output system 12 illuminates the field of view including the target 22 in a controlled manner, facilitating the capture of useful data by the imaging detector for the purpose of nerve detection.

The light output system 12 includes one or more light sources 202, one or more reflectors 204 and lenses 206, one or more wavelength filters 208, and a polarization system 210, the order and the number of each component above which may vary from what is presented here. The one or more light sources 202 dynamically produces light. Further, wherein the light source dictates characteristics such as intensity and wavelength spectrum.

When using a plurality of light sources in the light output system 12, each light source 202 may operate independently to emit light with their own set of lenses and filters, or a beam-joining system may be employed to emit light from a common output. The beam-joining system may make the imaging device more complex, also larger and heavier. Such a system may have the advantage that lights are emitted from the same output, reducing variations in glare and reflections when capturing the different images, which can have a negative impact in accuracy of the image processing algorithms.

The categories of light sources 202 that may be used include local sources and transmitted sources.

Local sources are compact and produce light close to the remaining aforementioned components of the light output. The common examples are high-power Light Emitting Diodes (LEDs) and laser diodes. These types of sources are less expensive and simpler to use, but they produce heat within the imaging device requiring challenging thermal management in a compact space.

Transmitted sources are larger and produce light in a separate location away from the imaging device, and the light is transmitted to the imaging device through a waveguide, such as a fiber optic cable or liquid light guide. The common examples are halogen lamps, laser tubes and higher power LEDs. These systems are more complex with more components, and are hence more expensive. However, since the light is produced away from the imaging device, thermal management is easier. It can therefore enable the imaging device itself to be more compact.

Depending on the type of light source 202, the light produced is most unfocussed, thereby wasting light energy directed to areas that are not being actively imaged. A system of reflectors 204 and/or lenses 206 focus or collimate the light to direct the majority of the light towards the intended target. This enables more energy to reflect off of the target tissue and reach the imaging detector in a focused manner, enhancing the quality of data. The reflectors 204 reflects efficiently the lights towards the target in a focused manner, and the lenses 206 may have an adjustable focusing system that adjusts angles of spread of the lights match the field of view. Wherein the angle of spread of the light may be adjusted to match the field of view of the imaging detection system 14 for optimal performance.

One or more wavelength filters 208 restricts the lights to specific wavelength spectrum to match the wavelengths of the imaging detection system 14. The filters and lenses may be electrically and/or mechanically tunable. A polarization system 210 creates a light output with adjustable angles of polarization depending on the formfactor and exacts use case in which this technology is being applied.

When using a plurality of light sources, each light source 202 works independently to emit light with their own set of lenses and filters, or a beam-joining system may be employed to emit light from a common output.

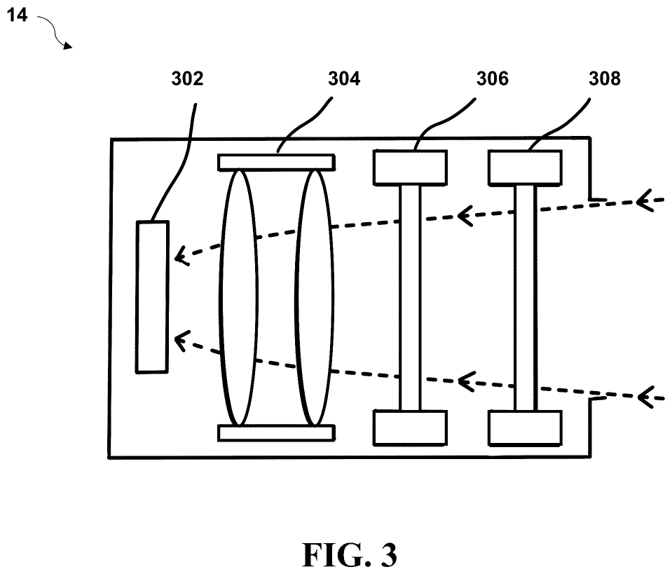

Referring now to FIG. 3, shown therein is a block diagram of an imaging detection system 14 for dynamic polarized light imaging, according to an embodiment. The specific arrangement, configuration, form, dimensions (e.g. shape, size), layout of the components of the imaging detection system 14 may be subject to variation depending on the application. The imaging detection system 14 records the light reflected from the target 22 being imaged, and captures a plurality of images of the target at different polarization angles in the field of view.

The imaging detection system 14 includes one or more imaging sensors 302, one or more lenses 304 that focus the light onto the imaging sensor 302, one or more wavelength filters 306 and a polarization system 308 that enables capture at different polarization angles. The order and the number of each component above which may vary from what is presented here.

When using a plurality of imaging sensors in the imaging detection system 12, each imaging sensor 302 may independently capture the polarized light from the field of view with their own lenses 304 and wavelength filters 306, or a beam-splitting system may be employed to capture light from a single point-of-view and directed to multiple imaging sensors. The beam-splitting system makes the imaging device more complex, also larger and heavier. Such a system has the advantage that all imaging sensors capture images from the same point-of-view, reducing parallax errors and the need for more intensive image registration.

Further, the plurality of imaging sensors 302 configured in the image detection system 14 may be designed for different light wavelengths such as visible, ultraviolet and infrared, in order to combine the data from these for more reliable nerve detection. The imaging sensor 302 may be a camera or similar, such as a Complementary Metal-Oxide-Semiconductor (CMOS) or a Charge-coupled device (CCD) that can capture images in grayscale or color.

The lenses 304 may be fixed or adjustable, and adjustably focus the polarized lights onto the plurality of imaging sensors 302 to view the target at different distances. The wavelengths filters 306 tunes to different light wavelengths, such as infrared using an IR bandpass filter or visible using an IR-UV cut filter. The polarization system 308 in the image detection system 14 executes individually and differs from the polarization system 210 in the light output system 12. The one or more lenses 304 and filters 306 may be electrically and/or mechanically tunable.

Referring now to FIGS. 4A-4D, shown therein are example methods for dynamically changing polarization for the light output system and the imaging detection system, according to an embodiment. In some embodiments, variations or combinations of approaches that may be taken for dynamically changing polarization for the light output system 12 and the imaging detection system 14 respectively depending on the application.

The approaches of the polarization system include discrete polarization angle approaches such as: separate polarized sources/sensors, polarimetric imaging sensors, and swappable polarization filters, and continuous variable polarization angle approaches such as: electrically tunable polarization filters and mechanically adjustable polarization filters.

Discrete polarization angle includes the polarization system (210 or 308) producing light or capturing images at certain discrete steps of polarization angles, and this may be cycled through during the imaging process.

Separate polarized sources/sensors 40 (FIG. 4A) includes the system using a set of distinct light sources or imaging sensors 402, each with its own independent linear polarization filters 404, fixed at distinct polarization angles. The active light output may be switched electrically and/or mechanically, and the polarization of the light depends on which output is active. The various outputs may have their own independent light source (such as separate LEDs) or a common light source may be directed simultaneously or sequentially to the different outputs. Images may be captured from the imaging sensors simultaneously or sequentially.

Polarimetric imaging sensors include the imaging sensor having pixel-level polarization filters and is able to capture images at multiple preset polarization angles simultaneously using a single sensor. An example is the Sony Polarsens® series of imaging sensors that capture images at 0°, 45°, 90° and 135° polarization angles simultaneously. The imaging system can employ a sensor like this.

Swappable polarization filters 42 (FIG. 4B) includes the system using a set of fixed polarization filters 406, fixed at distinct polarization angles, that may be cycled electrically and/or mechanically, through the optical path of the light source or imaging sensor 402 to achieve specific different polarization angles. The mechanism to swap the filters can in turn be driven by an electrically-controlled actuator such as a motor or a solenoid.

Continuous variable polarization angle includes the polarization system (210 or 308) producing light or capturing images at any variable polarization angle, and a specific subset of angles is cycled through during the imaging process.

Electrically tunable polarization filters (FIG. 4C) includes the system using adjustable polarization filter 408 based on liquid crystal or similar technology whose polarization angle may be adjusted within a specified range based on an applied electrical signal. The filter is placed in the optical path of the light source or imaging sensor 402 and the polarization of the light depends on the setting applied on this filter. This approach may not have any moving parts.

Mechanically adjustable polarization filters (FIG. 4D) includes the system using a fixed polarization filter 410 that may be mechanically rotated or translated in the optical path of the light source or imaging sensor 402 to adjust polarization angle of the light. The mechanical adjustment can in turn be driven by an electrically-controlled actuator such as a motor.

Referring now to FIGS. 5A-5C, shown therein are example methods for capture synchronization between the light output system and the imaging detection system, according to an embodiment. In some embodiments, variations or combinations of approaches that may be taken for capture synchronization between the light output system 12 and the imaging detection system 14 depending on the application.

As described in FIG. 1, the light output system 12 and the imaging detection system 14 are synchronized. This means that as the light output system 12 is dynamically adjusted, the image detection system 14 may also need to be adjusted (depending on the specific choice of detector system) to match the light output. The synchronization involves matching of the light polarization angle and possibly the field of view, focal length, and/or light wavelengths.

An aspect is synchronizing the polarization angle. As an example for the specific application of imaging nerves, the relative difference of the polarization angle of the light output and detector are orthogonal. Furthermore, data is captured at different polarization angles to detect all of the nerves in the field of view. Therefore, as the polarization angle of the light output 12 changes, the polarization angle of the image detector 14 may also change to maintain the orthogonal relationship.

To facilitate the synchronization, as well as to manage the data capture, the system 10 employs a capture synchronization and control system 16, this software control system may be running on an embedded microprocessor on an imaging device. This control system 16 manages the electrical and electromechanical adjustments available on the light outputs and detectors, as well as triggers data capture from the imaging sensors at the appropriate time. The control system 16 correlates the data captured with the settings on the outputs and detectors, and forwards the data sets to the image processing system 18.

The synchronization of the light output system 12 and the image detection system 14 may be accomplished by the capture synchronization and control system 16 using any one or more of electrically linked, mechanically linked, and software controlled.

Electrically linked 50 (FIG. 5A) includes the electrically adjustable components of the light output system 12 and the image detection system 14 may be electrically linked such that any signal to adjust one, automatically adjusts the other by the appropriate amount. For example, in case of electrically tunable polarization filters 502, the output and detector filters may be situated such that they are orthogonal at the zero-degree setting, and when their input signals are linked 504, they may maintain this orthogonal relationship throughout the adjustable angles.

Mechanically linked 52 (FIG. 5B) includes the mechanically adjustable components of the light output system 12 and the image detection system 14 may be mechanically linked (using gears, pulleys, linkages, or similar) such that the desired relationship between them is maintained as the component is adjusted. For example, in case of mechanically adjustable polarization filters 506, output and detector filters may be linked using a timing pulley system 508 or gears such that the filters are always orthogonal as they are rotated.

Software controlled 54 (FIG. 5C) includes the adjustable components of the light output system 12 and the image detection system 14, such as tunable polarization filters 502, may independently be under the direct control of the software control system 510 that takes on the responsibility of maintaining the appropriate relationship. This setup provides the most flexibility as the software can alter the relationship if needed, unlike electrical or mechanical linking. However, a software linked system may be more prone to errors and drift, and may require more independent monitoring systems and controllers.

Additionally, the data captured from the image sensors 302 are precisely synchronized with the settings of the light output system 12 and the image detection system 14 at the moment when the data is captured. The polarization angle of the light output system 12 is known when a given image is captured. This information is used by the image processing system 18 and correlates the intensity of the light detected from objects in the field of view to the angle of polarization of the light output system 12 and the image detection system 14.

In order to be aware of the polarization angle, the capture synchronization and control system 16 may have to use a feedback loop with independent monitoring sensors depending on the approach used to change the polarization angle. Monitoring sensors are not typically needed since the control system may specifically direct the transition to each discrete polarization angle. Hence the control system may inherently know the active polarization angle.

In case of continuous variable polarization angle approaches, monitoring sensors may be used. For example, in case of electrically tunable polarization filters, the control system may monitor the voltage of the signal applied to the filters as the voltage controls the angle. In case of mechanically adjustable polarization filters, the capture synchronization and control system 16 may need input from sensors like rotary encoders, angular potentiometers, hall-effect sensors, limit switches and/or photo-interrupts to monitor the exact angle of the filter when an image is captured.

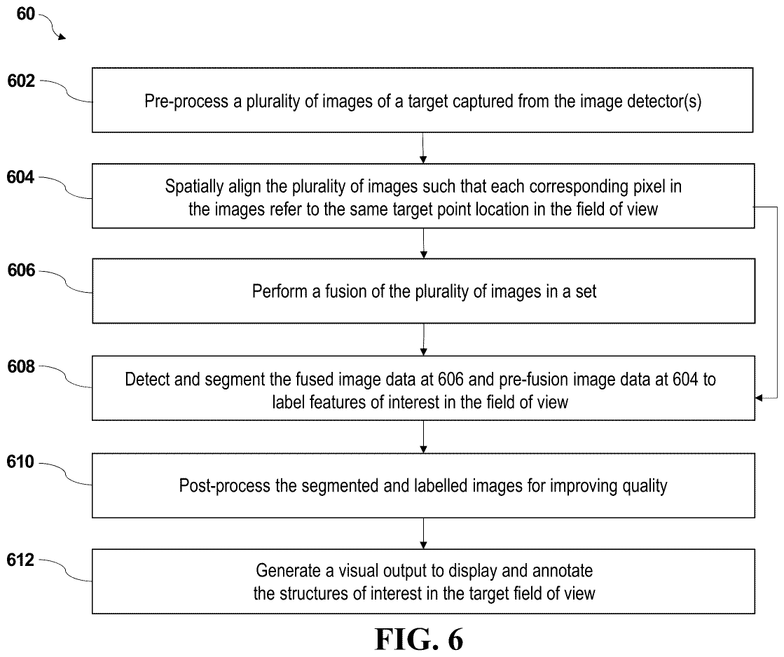

Referring now to FIG. 6, shown therein is an example method 60 for imaging processing, in particular, applied in an image processing system 18, according to an embodiment.

At 602, pre-processing a plurality of images of a target captured from an image detector 14. Pre-processing may include any combination of the following, but not limited to, brightness correction, contrast enhancement, histogram correction, cropping and scaling, distortion correction, color correction, noise reduction, image sharpening, averaging and filtering. The purpose of pre-processing is to potentially improve the quality of the images to obtain better results.

At 604, spatially aligning the plurality of images such that each corresponding pixel in the images refer to the same target point location in the field of view. The data from the capture synchronization and control system 16 and/or the motion tracking system 732 may be used to increase the accuracy of spatial alignment and speed up the process. The data captured from the supplementary imaging modalities 734 may also be spatially aligned with the image data for further processing.

At 606, performing a fusion of the plurality of images in a set. The angle of polarization at which the images are captured is taken into consideration when fusing the data in order to highlight structures of the target at different angles in the field of view. Machine learning techniques may also be employed to fuse the data.

At 608, detecting and segmenting the fused image data at 606 and pre-fusion image data at 604 to label features of interest in the field of view. Different machine learning models and/or conventional image processing steps may be configured to take the fused data as well as the pre-fusion image data to identify and label the various structures of interest. One or more types of structures in the field of view may be labelled depending on the application. The labeling may also have a confidence level attached to it that may be communicated to the user (graphically or textually) to indicate the certainty that a given labelling is accurate. Machine learning techniques may also be employed to extract features of interest.

At 610, post-processing the segmented and labelled images for improving quality and accuracy for the application. Post-processing involves various algorithms such as filtering and removal of incorrectly labelled areas (using properties like size, shape, etc.), averaging of multiple samples to improve confidence level, smoothening of labelled regions, etc. depending on the specific application.

At 612, generating a visual output to display and annotate the structures of interest in the target field of view. Further, the visually-appealing final output is generated to display the information desired by the user in an intuitive format. The approaches of displaying the final output includes, such as, taking the original image, and using transparent color overlays to highlight specific structures of interest. Other graphical or textual annotations may also be added, such as specifying size measurements or confidence level. This final output may be ultimately what is displayed to the user, for example on a monitor screen.

Referring now to FIG. 7, shown therein is an example method 70 for imaging processing, according to an embodiment. The image processing system 18 analyzes the data from the detector in real-time to display which tissues in the field of view are likely to be nerves. Image processing involves multiple steps, and may include a plurality of image processing approaches as well as machine learning based approaches. The steps may be performed in a different order than what is described herein, and some steps may be skipped, or additional steps included depending on the specific application.

The sequence of a plurality of images of the same target captured at different polarization angles and/or wavelengths forms one image set that is processed as a group to compute the final output.

A single image may be a part of more than one set, such as in a moving window grouping. As an example, with three sequential images at 0°, 120° and 240° polarization angles make a set, the next image captured in sequence may be an image back at 0° polarization angle. Now, the oldest image at 0° angle may be dropped and the moving 3-image window grouping may include the previous 120° and 240° images and the new 0° image to form a new set. This technique effectively triples the rate at which the output may be updated, instead of waiting for a fresh set of 3 images to recalculate the output.

Image processing may involve steps applied on individual images as well as steps applied on the image set as a whole. In case of a moving window grouping approach described above, the steps applied on a single image may be done only once and the results saved for reuse in the next set to improve computational efficiency.

At 702, pre-processing a plurality of images captured from the image detection system 14.

At 704, pre-processing a plurality of image data from supplemental modalities 734.

Both 702 and 704 may include any combination of the following image correction and enhancement, but not limited to, brightness correction, contrast enhancement, histogram correction, cropping and scaling, distortion correction, color correction, noise reduction, image sharpening, averaging and filtering. The purpose of pre-processing steps 702 and 704 is to potentially improve the quality of the images to obtain better results at subsequent steps.

At 706 and 708, a plurality of captured images in an image set may be registered or spatially aligned to ensure that each corresponding pixel in the images refers to the same structure in the field of view. Further, the data from the capture synchronization and control system 16 and/or from the motion tracking system 732 may be used to increase the speed and accuracy of image registration. Registration is critical as subsequent steps may compare the manner in which the data at a certain pixel varies from one image to another to make determinations about the structure at that pixel, such as its behavior at different polarization angles or wavelengths of light. Different algorithms may be employed to perform image registration process.

After 706 and 708, fusion of the image data may be performed in one or more stages. For example, the plurality of images in the image set from the image detection system 14 is fused at 710, and the data from the supplemental modalities 734 may be fused at 712. Different algorithms may be employed to fuse the images based on the data from the capture synchronization and control system 16. The angle of polarization at which the images are captured is taken into consideration when fusing the data in order to highlight structures at different angles in the field of view. Machine learning techniques may also be employed to fuse the data and extract features of interest.

At 714, the fused image data at 710 and 712 as well as the pre-fusion image data at 706 and 708 may be segmented and labelled to detect a plurality of features and the various structures of interest in the field of view. Different machine learning models and/or conventional image processing steps may be configured to take the fused data as well as the pre-fusion image data to identify and label the various structures of interest. One or more types of structures in the field of view may be labelled depending on the application. The labeling may also have a confidence level attached to it that may be communicated to the user (graphically or textually) to indicate the certainty that a given labelling is accurate.

At 716, the segmented and labelled images may be post-processed by checking labelled areas to improve the accuracy for the application. Post-processing involves various algorithms such as filtering and removal of incorrectly labelled areas (using properties like size, shape, etc.), averaging of multiple samples to improve confidence level, smoothening of labelled regions, etc. depending on the specific application.

At 718, generating a final output with the annotated the structures of interest in the field of view. At 720, displaying the final output. the approaches of displaying visually-appealing final output includes, such as, taking the original image, and using transparent color overlays to highlight specific structures of interest. Other graphical or textual annotations may also be added, such as specifying size measurements or confidence level. This final output may be ultimately what is displayed to the user, for example on a monitor screen.

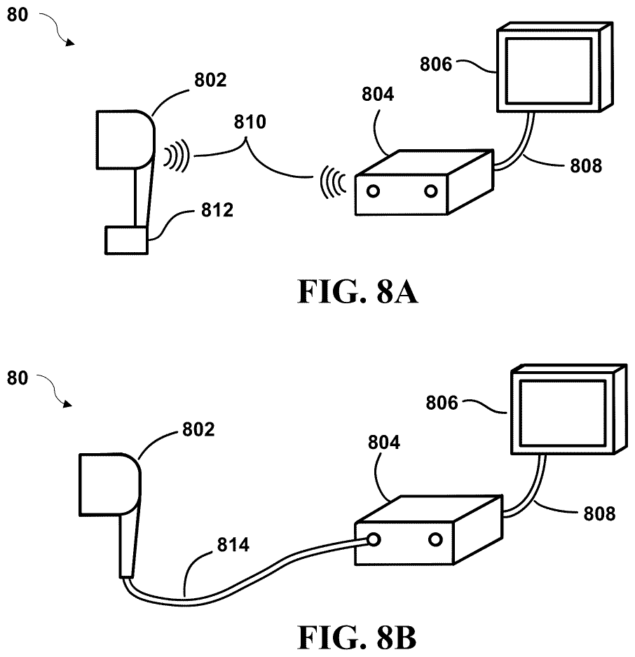

Referring now to FIGS. 8A-8B, shown therein are example functional components of the full system 80. The specific arrangement, configuration, form, dimensions (e.g. shape, size), layout of the components of the system 80 may be subject to variation depending on the application. The system 80 may be typically set up in an operating room. The system 80 may include an imaging device, which may be powered via a rechargeable battery system, a wired power source, or both to provide flexibility.

The choice of power source may be guided by the specific application. For example, battery power may be a better choice for a hand-held device, whereas wired power works better in a robot-assisted surgery application.

Battery-powered (FIG. 8A) includes the imaging device 802 having an integrated or an attached rechargeable battery 812 that may be capable of running the device for a specified period of time. Battery systems trade operation time limits and extra weight for the freedom of not being restricted by a wire. The battery may be recharged using one or more of several possible options such as wireless charging, wired charging, or swappable batteries.

Wireless charging includes the imaging device 802 wirelessly charging by placing at or near a charging station. The imaging device 802 may include the inductive power transfer coils and electronics for wireless charging. This technique has the benefit of not requiring exposed electrical contacts or connectors, making it easier for the imaging device to be made waterproof if desired.

Wired charging includes the imaging device 802 charging by plugging in a charger or placing into a charging station that makes electrical contact. This system provides more efficient energy transfer and faster charging.

Swappable battery includes the imaging device 802 allowing for the battery to be easily swapped with a fully charged one, and the discharged battery may be charged using a wireless or wired charging system. This system allows for fastest return-to-use once the battery is discharged, however, it introduces more separate parts in the entire system, such as the extra batteries that need to be handled and stored.

Wire-powered (FIG. 8B) includes the imaging device 802 being electrically connected to a power source with a wire 814. This technique has the benefit of unlimited run-time, and a lighter device without batteries. The power cable may be conveniently combined with the data cable, if applicable. With wired connections, the device may employ the appropriate rugged and reliable connectors for the specific application.

The imaging device 802, may be paired with a base station 804 to make the full system 80 (FIGS. 8A and 8B). This base station can serve one or more of various purposes depending on the specific design and application of the imaging device, such as image/data processing, power source, charging station, light source, data output and data recording.

Image/data processing includes running the image and data processing algorithms to extract information about the object being imaged. Image processing by fusing images from multiple different polarization angles may be rather computationally intensive. Such algorithms, in most cases, may not efficiently run in the small-scale embedded microprocessors that may be integrated within the imaging device. Therefore, the processing software may be run on a larger, faster, and higher-power computational system that may be located in the base station that has more space. The base station 804 may be directly plugged into the electrical mains, allowing for a significantly higher power availability for the computational system, as well as better cooling. The computational system may communicate with the imaging device to receive the imaging data, process it, and then produce the output with the desired objects in the field of view labelled/highlighted.

Power source includes where the imaging device 802 is wire-powered, the base station 804 acts as the power source for the device. The base station 804 may contain the power conversion system to generate the correct voltage and current for the imaging device 802 from the mains supply. The power lines to the imaging device 802 can accompany the data lines in a single cable 814.

Charging station includes where the imaging device 802 is battery-powered, the base station 804 can also serve as the charging station for the image device 802 and/or the removable batteries. The base station 804 can also provide for storage of the extra batteries and the imaging device while not in use. Alternatively, the charging and storage may be in a separate box outside the base station 804.

Light source includes where the imaging device 802 is using a transmitted light source, the base station 804 forms a possible location to place the light source inside. The base station 804 has the space, power and cooling to accommodate more powerful sources like halogen lamps and laser tubes. This can then be transmitted to the imaging device 804 using fiber optic or similar waveguides. Alternatively, the light source may be in a separate box outside the base station 804.

Data output includes where the base station 804 being the site of image processing, may also serve as the location from where the processed data may be output. In most cases, this may take the form of a display output port, such as Serial Digital Interface (SDI), High-Definition Multimedia Interface (HDMI), Digital Visual Interface (DVI), Video Graphics Array (VGA), or similar, to which a monitor or screen may be connected. The output of the system (such as images or videos with the desired objects labelled) may be presented visually on the display. The base station 804 may also provide for other forms of data output, such as USB and ethernet, to connect to other devices and computers for further data handling and/or recording.

Data recording includes where the base station 804 provides for data recording functionality. It may contain an internal storage medium, such as a hard disk drive, a solid-state drive, or a flash drive, that can record a certain duration of data on request. This data may be then reviewed and/or copied out if desired.

The imaging device 802 and the base station 804 may require robust communication. The communication approach may need to support high data throughput of the high-resolution images captured at high framerates (around 60 frames per second or higher or lower depending on application). It may also have low latency to ensure that there is minimal lag time between image capture and the processed data output.

There are two broad approaches for the communication: wireless (FIG. 8A) and wired (FIG. 8B). Wireless may be more convenient for the user, whereas wired communication is more robust. The choice may depend on the specific application. For a battery-powered imaging device, the communication may be wireless 810 so that the device is tether-free. For a wire-powered imaging device, the communication may be wired since there is already a tether 814 to the base station 804.

The communication design may also involve the selection of a communication protocol. The protocol may be a standard one, such as USB or ethernet for wired, or Bluetooth or Wi-Fi for wireless. The protocol may also be a customized one, developed specifically for this system to have better dedicated performance. A customized protocol can make communication more efficient by reducing unnecessary overhead and/or more reliable by improving error handling and noise immunity. Any wireless protocol may have to be in the gigahertz frequency range to support the high data throughput.

The final output of the image processing system 18 may be displayed to the user on a monitor screen 806 or similar. The base station 804 may be where bulk of the image processing is performed, and the display output may thus be provided by the base station 804.

The base station 804 may have a wired connection 808 to the monitor screen. The base station 804 may support multiple different display output formats for compatibility with different monitors, including but not limited to Serial Digital Interface (SDI), High-Definition Multimedia Interface (HDMI), Digital Visual Interface (DVI), Video Graphics Array (VGA), S-Video, Composite Video, and Component Video.

The monitor screen 806 may be provided as an integrated part of the full system 80 described herein, such as on a cart along with the base station 804, or integrated into the base station 804.

Alternatively, the base station 804 may be connected to an external display, television, or monitor, such as those often pre-installed in an operating room. It is also possible to have a small display on the imaging device 802 itself, so that a separate screen is not needed.

The base station 804 may also provide additional connections for various purposes depending on the application. This may include interfaces for an external recording system, for connecting to internet or other imaging hardware, and for debugging.

The user interface to interact with the system 10 may take many different forms depending on the application. It is also possible to have multiple redundant user interface options on the same system for added flexibility. User interface options include, but are not limited to: buttons or touchscreen on the imaging device, buttons or touchscreen on the base station, external touchscreen monitor, a remote-control dongle, and motion gesture recognition. The system 10 may also include microphones to listen and respond to voice commands. The user interface may include multiple indicator lights for visual feedback, speakers for auditory feedback, and vibration motors for haptic feedback. The user interface may provide the user with ability to control various functions of the system that may include: standby mode, recording start/stop, zoom, focus, white balance, display modes, imaging modes, display brightness/contrast, and image processing adjustments.

Machine learning and neural-network based image processing can also be applied to further improve the accuracy of detecting the nerves in the field of view. Machine learning models may be effective in combining the image data from multiple forms of imaging modalities, such as dynamically-changing polarized light and varying wavelength spectrums.

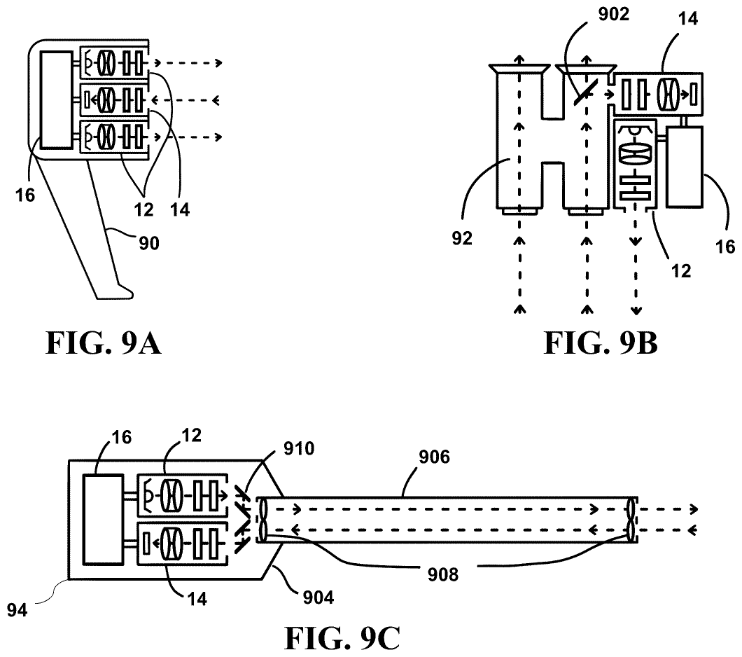

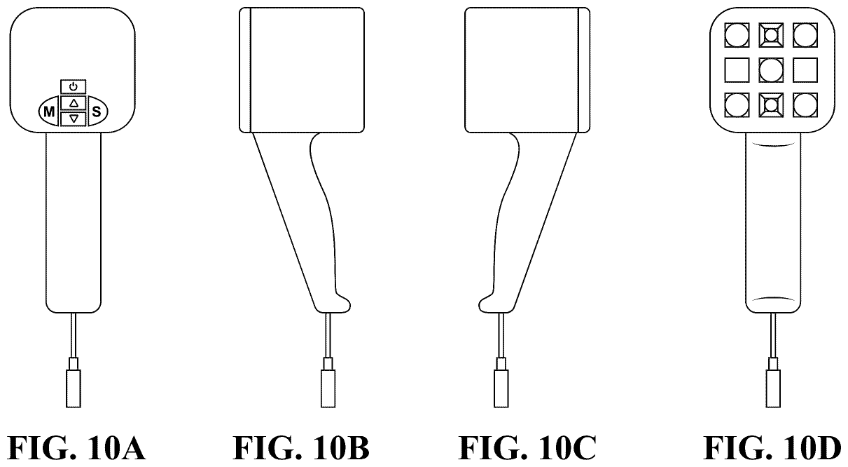

Referring now to FIGS. 9A-9C and 10A-10D, shown therein are example devices employing the light output system 12, the imaging detection system 14 and the capture synchronization and control system 16 for dynamically changing polarized light imaging, according to an embodiment.

FIG. 9A shows an example device of this invention for use in open surgery. The device may be fashioned as a handheld or a handsfree (mounted on an adjustable arm or similar) device, akin to a barcode scanner, similar to FIG. 10A-10D.

The body of the device 90 may include a head and a handle. The head of the device may contain the light output system 12 and the imaging detection system 14 controlled by the capture synchronization and control system 16. The handle may be ergonomically designed to provide a secure and comfortable grip, and may be angled forward to facilitate the user in directing the device downward towards the surgical field with ease. The device 90 may be able to function from a distance, such as, around 20 cm to 30 cm, when pointed at the surgical field.

Notably, the handle may form an acute (<90 degrees) angle with respect to the head, as shown in FIGS. 9A, 10B and 10C. This is in contrast to most handheld devices in a similar formfactor, like barcode scanners, where the handle forms an obtuse (>90 degrees) angle with respect to the head. This acute angle design allows for the device to be more comfortably held in a position that points the head in a downward angle towards the surgical field.

The body of the device may include a provision for secure attachment to an adjustable arm or stand for handsfree usage. This may permit the device 90 to be used in handheld mode or handsfree mode depending on the preference of the user or the needs of the application.

FIG. 10A shows a back view of the body of device 90 and illustrates a plurality of buttons and indicator lights. The plurality of buttons at least includes 5 buttons for controlling the device, 1 button for power on/off and 4 buttons for changing the settings of the device 90. The functionality of each button may vary based on the specific application. The plurality of indicator lights at least includes 2 on either side of the power button for presenting different information to the user. The functionality of each indicator light above may vary based on the specific application.

FIG. 10D discloses a front view of the head of device 90. In this embodiment, the light output system 12 comprises the infrared (IR) light sources with dynamically changing polarization located at the four corners of the front view of the head, using mechanical rotation of the polarizing filter approach in this prototype. In this embodiment, the imaging detection system 14 comprises an IR camera located at the center of the front view of the head, also with dynamically changing polarization, which is mechanically synchronized to the light sources to maintain the 90° offset in this prototype.

Additionally, the middle at the top of the front view of head may be a white light source (for the visible spectrum camera), and the middle at the bottom of the front view of head may be a visible spectrum RGB camera. In this prototype, the white light source and the visible spectrum RGB camera are not employing dynamically changing polarization. The RGB camera may be used to capture a full color video of the target such that the structures of interest may be identified using dynamically changing polarized light imaging and then highlighted on this color video to form the final output of the system.

Various design considerations may be evaluated when developing the body of the device. The body of the device 90 allows for the cleaning and disinfection procedures for surgical equipment, which may necessitate it being waterproof. The materials may be biocompatible as well as rugged. The device 90 may also provide adequate thermal management for its components to ensure reliable performance without overheating. This may be a challenge as the device may not be able to have air vents due to waterproofing. In case of a sealed system, thermal management may need to be accomplished by conducting heat across the body enclosure, thereby making thermally conductive materials like metals a better choice for the enclosure. Total weight as well as weight distribution are also considerations for ergonomics.

In situations where the device itself cannot be sterilized (due to temperature or material restrictions), the device may include the provision to snap on a disposable or reusable sterile drape that covers the device in its entirety, as well as a length of the cable in case of a wired device. This covers the unsterilized device in a sterile field and permits safe use in the operating room. The drape may be specifically designed for the device to ensure a proper fit, as well as provide a transparent viewing window through which the device can operate and perform imaging.

FIG. 9B shows an example device, which is attached to a surgical microscope which is used for microscopic open surgeries. A device 92 with a surgical microscope setup intends to provide a magnified view of a smaller surgical field compared to conventional open surgery. This allows for microsurgical procedures involving delicate structures like small vessels and nerves. Attached to the microscope may be the light output system 12 and the imaging detection system 14 controlled by the capture synchronization and control system 16.

Attaching the device 92 to the microscope permits handsfree operation, and the device 92 can follow the field-of-view and focus of the microscope. The final display output may be presented on an external monitor, or could be integrated into the microscope eyepiece to provide an augment-reality experience where the structures are highlighted in the microscope's view. The device 92 may be attached onto an existing microscope as a retrofit (such as simply attaching it to a side and pointing to the same field-of-view as the microscope), or may be integrated into the microscope and potentially share some of the optics.

In FIG. 9B, the light output system 12 is attached onto the side of the microscope and pointed to the same field-of-view, as one attachment option. On the other hand, the imaging detection system 14 is integrated into the microscope and shares the same optical path as the microscope's objective by using a beam-splitting mirror 902, as another attachment option. The light output system 12 and imaging detection system 14 may use either attachment approach depending on the application.

FIG. 9C shows an example device of integration into an endoscope 94 for use in minimally invasive surgeries, such as an endoscope-integrated device for minimally invasive manual or robotic surgery. The imaging technology described herein may be packaged in a compact format for use in the endoscope 94. Endoscope 94 has a long narrow shaft 906 that enters the body through small incisions to provide a view of the inside of the body, while the larger head 904 remains outside the body. Endoscope 94 may be used for both manual and robotic minimally invasive surgeries with some structural design adjustments. The head 904 of the endoscope 94 may contain the light output system 12 and the imaging detection system 14 controlled by the capture synchronization and control system 16.

A system of mirrors 910, prisms, beam-splitters and/or lenses 908 may be used to guide the outgoing and incoming beams of light close together such that they may be directed into the long narrow shaft 906 of the endoscope 94. The long narrow shaft 906 may have additional optics, such as lenses 908 and prisms, on either end for proper transmission of light as a collimated beam through the lumen of the shaft. Alternatively, the long narrow shaft 906 may employ fiber optics or other waveguides to transmit light along its length.

Parts of the light output system 12 and imaging detection system 14, such as polarization or wavelength filters, may be moved to the distal end of the shaft 906 of the endoscope 94. This approach would have the benefit of filtering the light closer to the target and avoid errors due to loss of polarization during transmission along the long narrow shaft 906.

With advancement in electronics and the availability of miniature cameras and miniature light sources (such as tiny bright LEDs), some variations are possible where the entire light output system 12 and imaging detection system 14 may be made small enough to be right at the distal end of shaft 906 of the endoscope 94, rather than the head 904 of the endoscope 94.

The depictions are simplified schematic layouts to represent the conceptual design, and specific device designs may vary.

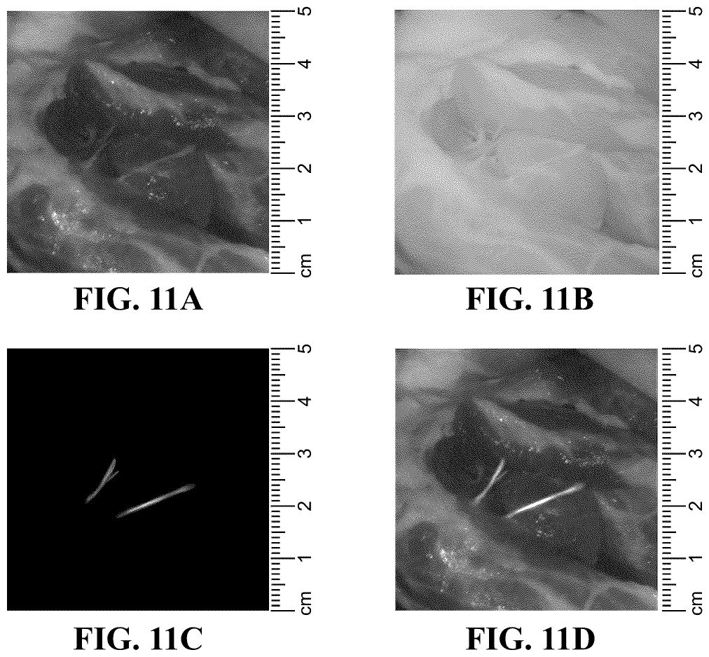

Referring now to FIGS. 11A-11D, shown therein are example raw captured images and final outputs highlighting the detected nerves, according to an embodiment. In FIGS. 11A-11D, the collected image data are sample data obtained from pig subjects that demonstrates the intended functionality of the device.

FIG. 11A illustrates an example of the raw visible light (RGB) images captured for further image processing by an example imaging device, such as a handheld imaging device (FIGS. 10A-10D).

FIG. 11B illustrates an example of the raw infrared (IR) images captured for further image processing by an example imaging device, such as a handheld imaging device (FIGS. 10A-10D).

FIG. 11C illustrates an example of the nerve detection masks computed as an intermediate step during image processing by an example imaging device, such as a handheld imaging device (FIGS. 10A-10D).

FIG. 11D illustrates an example of the color overlay images with the nerves highlighted for the display output by an example imaging device, such as a handheld imaging device (FIGS. 10A-10D).

The collected data includes both infrared (IR) and visible light (RGB) images, acquired using an example imaging device. Multiple IR images were captured at varying polarization angles, with only one such image presented (FIG. 11B) as an example. These IR images, in conjunction with the RGB images, are processed and analyzed to detect nerves. The detected nerves are then emphasized in the final output (FIG. 11D) by means of a color overlay.

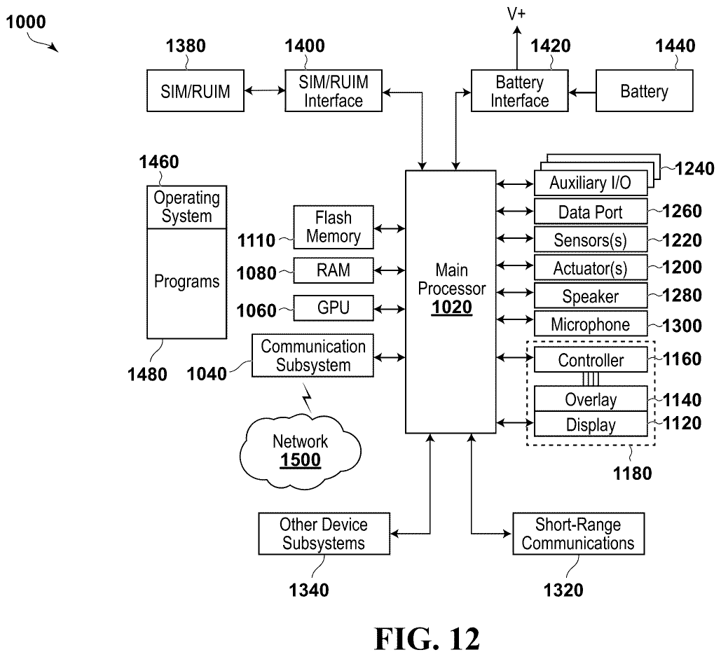

Referring now to FIG. 12, shown therein is a block diagram of a computing device 1000 used in the system 10 for dynamic polarized light imaging of FIG. 1, according to an embodiment. The computing device 1000 may be, for example, a part of any one or more of the components 12, 14, 16, 18 and/or 20 of FIG. 1.

The computing device 1000 includes multiple components such as a main processor 1020 that controls the operations of the computing device 1000. Communication functions, including data communications, voice communications, or both may be performed through a communication subsystem 1040. The communication subsystem 1040 may receive data from and send data to a wireless network 1500. The main processor may use a Graphics Processing Unit (GPU) 1060 to efficiently execute image and video processing tasks.

The wireless network 1500 may be any type of wireless network, including, but not limited to, cellular networks, Wi-Fi networks, and Bluetooth networks.

The computing device 1000 may be a battery-powered device and include a battery interface 1420 for receiving one or more rechargeable batteries 1440.

The processor 1020 also interacts with additional subsystems such as a Random Access Memory (RAM) 1080, a flash memory 1110, a display 1120 (e.g., with a touch-sensitive overlay 1140 connected to an electronic controller 1160 that together comprise a touch-sensitive display 1180), one or more actuators 1200, one or more sensors 1220, an auxiliary input/output (I/O) subsystem 1240, a data port 1260, a speaker 1280, a microphone 1300, short-range communications systems 1320 and other device subsystems 1340.

In some embodiments, user-interaction with the graphical user interface may be performed through the touch-sensitive overlay 1140. The processor 1020 may interact with the touch-sensitive overlay 1140 through the electronic controller 1160. Information, such as text, characters, symbols, images, icons, and other items that may be displayed or rendered on a computing device generated by the processor 1020 may be displayed on the touch-sensitive display 1180.

To identify a subscriber for cellular network access according to the present embodiment, the computing device 1000 may use a Subscriber Identity Module or a Removable User Identity Module (SIM/RUIM) card 1380 inserted into a SIM/RUIM interface 1400 for communication with a network (such as the wireless network 1500). Alternatively, user identification information may be programmed into the flash memory 1110 or performed using other techniques.

The computing device 1000 also includes an operating system 1460 and software components 1480 that are executed by the processor 1020 and which may be stored in a persistent data storage device such as the flash memory 1110. Additional applications may be loaded onto the computing device 1000 through the wireless network 1500, the auxiliary I/O subsystem 1240, the data port 1260, the short-range communications subsystem 1320, or any other suitable device subsystem 1340.

In use, a received signal may be processed by the communication subsystem 1040 and input to the processor 1020. The processor 1020 then processes the received signal for output to the display 1120 or alternatively to the auxiliary I/O subsystem 1240. The processor 1020 may also transmit data over the wireless network 1500 through the communication subsystem 1040.

For voice communications, the overall operation of the computing device 1000 may be similar. The speaker 1280 may output audible information converted from electrical signals, and the microphone 1300 may convert audible information into electrical signals for processing.

While the above description provides examples of one or more apparatus, methods, or systems, it may be appreciated that other apparatus, methods, or systems may be within the scope of the claims as interpreted by one of skill in the art.

1. A system for dynamic polarized light imaging, the system comprising:
a light output system including one or more light sources that dynamically produces polarized light with varying polarization angles, wavelengths and/or focus;
an imaging detection system including one or more image detectors that captures images of a target under varying polarization angles, wavelengths and/or focus;
a capture synchronization and control system that manages electrical and electromechanical adjustments of the light output system and the imaging detection system, to dynamically adjust the light sources and image detectors to synchronize with each other; and
an image processing system that processes a plurality of images captured to compute and produce a final output.

2. The system of claim 1, wherein the light output system includes a polarization system to vary the polarization angle of the light produced, using a discrete polarization angle approach or a continuous variable polarization angle approach.

3. The system of claim 1, wherein the light output system includes a plurality of reflectors to efficiently reflect the polarized light towards the target in a focused manner.

4. The system of claim 1, wherein the light output system includes a plurality of lenses to adjust angles of spread and/or focus of the polarized light match the field of view and/or distance to the target.

5. The system of claim 1, wherein the light output system includes a plurality of optical filters to restrict the polarized light to specific wavelength spectrum.

6. The system of claim 1, wherein the light output system includes a beam-joining system for emitting the polarized light from a common output.

7. The system of claim 1, wherein the image detection system includes a polarization system to vary the polarization angle of the light captured, using a discrete polarization angle approach or a continuous variable polarization angle approach.

8. The system of claim 1, wherein the image detection system includes a polarization system that differs from the polarization system in the light output system.

9. The system of claim 1, wherein the image detection system is configured to record reflected light with a distribution of polarization angles from the target being imaged in a field of view.

10. The system of claim 1, wherein the imaging detection system includes a plurality of optical filters for tuning to different light wavelengths.

11. The system of claim 1, wherein the imaging detection system includes a plurality of lenses that adjustably focus the reflected polarized light onto the plurality of imaging sensors to view the target at different fields of view and/or distances.

12. The system of claim 1, wherein the imaging detection system includes a plurality of imaging sensors for capturing multiple images that are fused and processed for detecting optically anisotropic structures of interest, such as nerves, in the field of view.

13. The system of claim 12, wherein the plurality of imaging sensors is configured to independently capture the reflected polarized lights from the field of view with their own lenses and optical filters.

14. The system of claim 1, wherein the imaging detection system further includes a beam-splitting system for directing light from a single point-of-view to the plurality of imaging sensors.

15. The system of claim 1, wherein the polarization system included in the imaging detection system is configured to capture a plurality of images of the target at different polarization angles.

16. The system of claim 1, wherein the capture synchronization and control system maintains the relationship between the polarization angles and other parameters of the light output system and the image detection system, using an approach related to electrical linking, mechanical linking, or software control.

17. The system of claim 1, wherein the system further includes an image processing system configured to process a plurality of images of the target captured in the field of view under different polarization angles and different light wavelengths, such that structures that are optically anisotropic appear with different brightness in the plurality of images.

18. The system of claim 1, wherein the image processing system is further configured to spatially synchronize the plurality of images for making determinations about the structure at a certain pixel from data captured, such that each corresponding pixel in the images refer to the same point in the field of view.

19. The system of claim 1, wherein the image processing system is further configured to segment and identify structures of interest in the field of view.

20. The system of claim 1, wherein the system further includes or connects to an output device for highlighting and displaying the structures of interest in the field of view.

21. The system in claim 1, wherein the final output that is displayed is configured to identify the structures of interest in the field of view using a color overlay or annotation, and further specify a confidence level to indicate the certainty that a given labelling is accurate.

22. The system of claim 1, wherein the light output system, the imaging detection system, and the capture synchronization and control system are integrated in an imaging device.

23. The system of claim 22, wherein the imaging device is a handheld device including an ability to optionally attach the imaging device to a hands-free mounting system.

24. The system of claim 23, wherein the handheld imaging device includes a handle that forms an acute (<90 degrees) angle with respect to the head, allowing for the handheld device to be more comfortably pointed down towards a surgical field.

25. The system of claim 22, wherein the imaging device is attached to or integrated into a surgical microscope.

26. The system of claim 22, wherein the imaging device is attached to or integrated into an endoscope.

27. The system of claim 1, wherein the system further includes a motion tracking system for tracking translational and rotational motion as well as a distance of the imaging device from the target being imaged.

28. The system of claim 1, wherein the capture synchronization and control system is further configured to execute a feedback loop with independent monitoring sensors to change the polarization angle, focus, or other imaging parameters in a precise and coordinated manner.

29. The system of claim 1, wherein the system is further configured to perform ambient light compensation by estimating an ambient light intensity to adjust for dynamic variations of ambient lighting conditions.

30. The system of claim 1, wherein the system is further configured to perform distance compensation by estimating a distance to a target when the distance of the light output system or the imaging detection system from the target is varied during use.

31. The system of claim 1, wherein the system is further configured to perform motion compensation by estimating the motion of the imaging device for spatially aligning the plurality of images when the imaging device is moved during use.

32. The system of claim 1, wherein the system is further configured to perform target surface orientation compensation using a plurality of light outputs and/or imaging detectors to capture images under different lighting perspectives.

33. The system of claim 1, wherein the system is further configured to integrate image data acquired by at least one manner of regular visible light color imaging, laser speckle contrast imaging (LSCI), near-infrared (NIR) imaging, hyperspectral imaging, fluorescent dye imaging, autofluorescence imaging, and Raman scattering imaging into the dynamic polarized light imaging, to identify and distinguish various structures.

34. A method for image data processing, the method comprising:
pre-processing a plurality of image data of a target captured in a field of view for image correction and enhancement by algorithms;
spatially aligning the plurality of image data for making determinations about structures of interest at a certain pixel from the data captured;
fusing the plurality of image data to detect varying brightness of pixels and highlight structures of interest at different angles in the field of view;
detecting and segmenting the fused image data and pre-fusion image data to label features of interest in the field of view;
tracking data related to translational and rotational motion, and distance of an imaging device from the target being imaged to support the alignment among captured images;
post-processing the segmented and labelled images for improving quality; and
displaying a visually-appealing final output with the structures of interest highlighted and labelled.

35. The method of claim 34, wherein the image data further includes data related to ambient light compensation, target distance compensation, and target surface orientation compensation to improve output accuracy.

36. The method of claim 34, wherein the method further includes providing additional data to improve output performance by integrating supplemental imaging modalities.

37. An imaging apparatus for dynamically changing polarized light imaging, the imaging apparatus comprising:
a handle for providing a grip to facilitate a user in directing the imaging apparatus downward towards a field during a surgery;
a head for employing a plurality of components, wherein the plurality of components further includes:
a light output system including a plurality of infrared (IR) light sources with dynamically changing polarization located at up to four corners of the front view of the head, using mechanical rotation of a polarizing filter approach, and a white light source in the middle at the top or bottom of the front view of the head;
an imaging detection system including an IR camera located at the center of the front view of the head, also with dynamically changing polarization, and mechanically synchronized to the light sources to maintain a 90° offset, and a visible spectrum RGB camera in the middle at the top or bottom of the front view of the head;
a capture synchronization and control system configured to manage electrical and electromechanical adjustments of the light output system and the imaging detection system, to dynamically adjust the light sources and image detectors to synchronize with each other; and
a plurality of buttons for supporting the user to control the imaging device, and a plurality of indicator lights for presenting information to the user.

38. A system for real-time image processing, the system comprising:
a base station configured to provide a plurality of functions, including image data processing, power source, charging station, light source, data output, and data recording, wherein the image data processing at least includes fusing images from the varying polarization angles, wavelengths and/or focus;
an imaging device configured to capture a plurality of images of a target in a field of view under the varying polarization angles and varying light wavelengths, such that structures that are optically anisotropic appear with varying brightness in the plurality of images and structures of interest are highlighted; and
wherein the base station is communicatively connected to the imaging device, and a plurality of display hardware.

39. A method for dynamic polarized light imaging, the method comprising:
producing dynamically changing polarized lights from at least one or more light sources;
capturing, by at least one or more image detectors, a plurality of images of a target and recording reflected light from the target being imaged in a field of view, wherein the reflected light includes a distribution of polarization angles;
managing, by a capture synchronization and control system, electrical and electromechanical adjustments available on both the light output system and the imaging detection system, in order to dynamically adjust the image detectors to synchronize with the light outputs; and
processing, by an image processing system, a plurality of images of the target under different polarization angles and different light wavelengths, such that structures that are optically anisotropic appear with varying brightness in the plurality of images and the structures of interest are highlighted.

40. A method for image processing for dynamic polarized light imaging, the method comprising:
spatially aligning a plurality of images;
executing a fusion of image data to detect varying brightness and highlight structures of the target at different angles in the field of view;
detecting and segmenting the fused and pre-fusion image data to label features of interest in the field of view;
post-processing the segmented and labelled images for improving quality; and
generating a visual output to display and annotate the structures of the interest in the field of view.
